(19) United States
(12) Patent Application Publication
(10) Pub. No.: US 2024/0257944 A1
(43) Pub. Date: Aug. 1, 2024

(54) MEDICAL INFORMATION PROCESSING APPARATUS, MEDICAL IMAGE DIAGNOSTIC APPARATUS, AND COMPUTER-READABLE NON-TRANSITORY STORAGE MEDIUM

(71) Applicant: CANON MEDICAL SYSTEMS CORPORATION, Tochigi (JP)

(72) Inventors: Takuya SAKAGUCHI, Utsunomiya (JP); Toru TAKAHASHI, Nasushiobara (JP); Shumpei OHASHI, Otawara (JP); Takahiko NISHIOKA, Otawara (JP); Ko FUCHIGAMI, Ota (JP)

(73) Assignee: CANON MEDICAL SYSTEMS CORPORATION, Tochigi (JP)

(21) Appl. No.: 18/425,003

(22) Filed: Jan. 29, 2024

(30) Foreign Application Priority Data
Jan. 30, 2023 (JP) 2023-012209

(51) Int. Cl. G16H 30/20

(52) U.S. Cl. CPC G16H 30/20

(57) ABSTRACT

A medical information processing apparatus according to an embodiment includes processing circuitry configured to acquire a total amount of a contrast agent available for a subject during a procedure, acquire an analysis result for each contrast imaging condition about the subject, and output information on use of the contrast agent based on the total amount of the contrast agent and the analysis result.

### CROSS-REFERENCE TO RELATED APPLICATIONS

This application is based upon and claims the benefit of priority from Japanese Patent Application No. 2023-012209, filed on Jan. 30, 2023; the entire contents of which are incorporated herein by reference.

### FIELD

Embodiments described herein generally relate to a medical information processing apparatus, a medical image diagnostic apparatus, and a computer-readable non-transitory storage medium.

### BACKGROUND

In procedures targeted for blood vessels, contrast images in which blood vessels of a subject are contrasted may be collected using a contrast agent. An x-ray diagnostic apparatus executes an x-ray scan on a subject injected with the contrast agent and can collect fluoroscopic images in which blood vessels are contrasted or collect angiographic X-ray images with higher image quality, for example. In addition, an X-ray computed tomography (CT) apparatus or a magnetic resonance imaging (MRI) apparatus can collect three-dimensional images in which blood vessels are contrasted.

### BRIEF DESCRIPTION OF THE DRAWINGS

FIG. 1 is a block diagram of an example of the configuration of a medical information processing system according to a first embodiment;

FIG. 2 is a block diagram of an example of the configuration of an X-ray diagnostic apparatus according to the first embodiment;

FIG. 3 is a diagram for illustrating a flow model according to the first embodiment;

FIG. 4 is a diagram for illustrating estimation of a contrast image according to the first embodiment;

FIG. 5 is a diagram for illustrating availability information of a contrast agent according to the first embodiment;

FIG. 6 is a diagram of an example of a blood vessel shape according to the first embodiment;

FIG. 7 is a diagram of a modification of the blood vessel shape according to the first embodiment;

FIG. 8 is a diagram for illustrating information on change to a contrast imaging condition under which a scheduled use amount of the contrast agent according to a second embodiment can be achieved;

FIG. 9 is a diagram for illustrating the information on change according to the second embodiment;

FIG. 10 is a diagram of a display example of the information on change according to the second embodiment;

FIG. 11 is a diagram of a display example of the information on change according to the second embodiment;

FIG. 12 is a diagram for illustrating flow velocity and diffusion of the contrast agent according to the second embodiment;

FIG. 13 is a diagram of an example of processing according to a third embodiment; and

FIG. 14 is a diagram for illustrating washout ability according to another embodiment.

### DETAILED DESCRIPTION

A medical information processing apparatus according to embodiments comprises processing circuitry configured to acquire a total amount of a contrast agent available for a subject during a procedure, acquire an analysis result for each contrast imaging condition about the subject, and output information on use of the contrast agent based on the total amount of the contrast agent and the analysis result.

The following describes embodiments of a medical information processing apparatus, a medical image diagnostic apparatus, and a computer-readable non-transitory storage medium in detail with reference to the accompanying drawings.

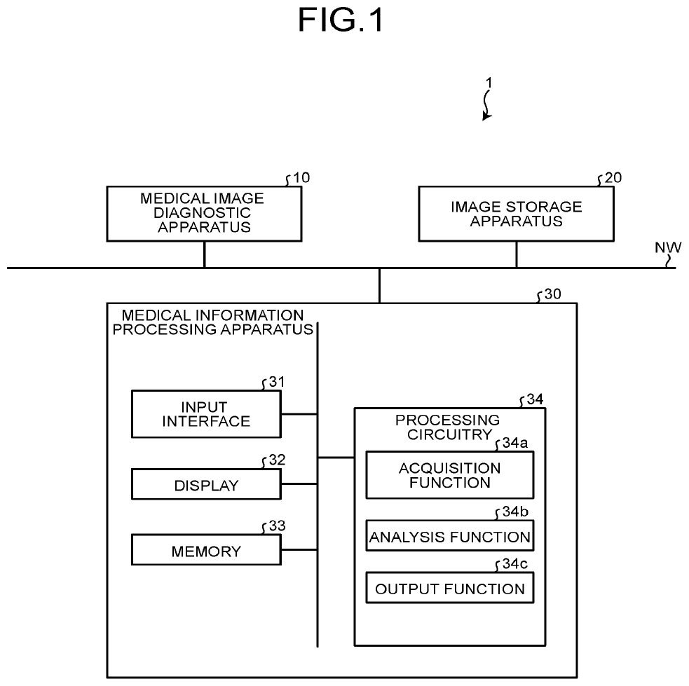

A first embodiment describes a medical information processing system 1 illustrated in FIG. 1 as an example. The medical information processing system 1 includes a medical image diagnostic apparatus 10, an image storage apparatus 20, and a medical information processing apparatus 30, for example. The medical image diagnostic apparatus 10, the image storage apparatus 20, and the medical information processing apparatus 30 are connected to each other via a network NW. FIG. 1 is a block diagram of an example of the configuration of the medical information processing system 1 according to the first embodiment.

The medical image diagnostic apparatus 10 is an apparatus collecting medical images from a subject. When a contrast agent is injected into the blood vessels of the subject, the medical image diagnostic apparatus 10 can collect medical images in which the blood vessels of the subject are contrasted. Specific examples of the medical image diagnostic apparatus 10 include an X-ray diagnostic apparatus, an X-ray computed tomography (CT) apparatus, and a magnetic resonance imaging (MRI) apparatus.

In the following, a medical image in which blood vessels are contrasted will also be referred to simply as a contrast image. The contrast image may be a two-dimensional image or a three-dimensional image. For example, when the medical image diagnostic apparatus 10 is an X-ray CT apparatus, it executes a CT scan on a subject with a contrast agent injected into blood vessels and can thereby collect projection data of a plurality of views in which the blood vessels are contrasted and reconstruct a three-dimensional X-ray CT image in which the blood vessels are contrasted. The contrast image may be a digital subtraction angiography (DSA) image with background components such as bone and soft tissue subtracted. Injection of the contrast agent into the blood vessels of the subject may be performed by an injector included in the medical image diagnostic apparatus 10 or manually by a user such as a doctor or medical professional.

The image storage apparatus 20 is an apparatus storing the medical images collected by the medical image diagnostic apparatus 10. For example, the image storage apparatus 20 is implemented by a computer device such as a server apparatus. Specifically, the image storage apparatus 20 acquires the medical images from the medical image diagnostic apparatus 10 via the network NW and stores the medical images in a memory provided inside or outside the apparatus.

Although FIG. 1 illustrates only one medical image diagnostic apparatus 10, the medical information processing system 1 may include a plurality of medical image diagnostic apparatuses 10. In this case, the image storage apparatus 20 stores medical images collected by the medical image diagnostic apparatuses 10. The medical information processing system 1 may include a plurality of kinds of modalities such as an X-ray diagnostic apparatus, an X-ray CT apparatus, and an MRI apparatus as the medical image diagnostic apparatuses 10. In this case, the image storage apparatus 20 stores various kinds of medical images such as X-ray images, X-ray CT images, and MRI images.

The medical information processing apparatus 30 has an input interface 31, a display 32, a memory 33, and processing circuitry 34. The medical information processing apparatus 30 outputs information on use of the contrast agent in the medical image diagnostic apparatus 10 by processing by the processing circuitry 34 described below and can make use of the contrast agent during a procedure more appropriate.

The input interface 31 receives various kinds of input operations from the user, converts the received input operations into electric signals, and outputs the electric signals to the processing circuitry 34. The input interface 31 is implemented by a mouse, a keyboard, a trackball, a switch, a button, a joystick, a touchpad performing input operations through touching on an operating surface, a touchscreen with a display screen and a touchpad integrated, a non-contact input circuit including an optical sensor, or a voice input circuit, for example. The input interface 31 may include a tablet terminal or the like that can wirelessly communicate with the processing circuitry 34. The input interface 31 may be a circuit receiving input operations from the user through motion capture. To give an example, by processing signals acquired via a tracker and images collected about the user, the input interface 31 can receive user's body movements, gaze, and the like as input operations. The input interface 31 is not limited to those including physical operating components such as a mouse or a keyboard. Examples of the input interface 31 include electric signal processing circuitry receiving electric signals corresponding to input operations from an external input device provided separately from the medical information processing apparatus 30 main body and outputting these electric signals to the medical information processing apparatus 30 main body.

The display 32 displays various kinds of information. The display 32 displays a graphical user interface (GUI) for receiving various instructions, settings, and the like from the user via the input interface 31, for example. The display 32 is a liquid crystal display or a cathode ray tube (CRT) display, for example. The display 32 may be of a desktop type or include a tablet terminal or the like that can wirelessly communicate with the medical information processing apparatus 30 main body.

In FIG. 1, the medical information processing apparatus 30 is described as including the display 32, but the medical information processing apparatus 30 may include a projector instead of or in addition to the display 32. The projector can perform projection onto a screen, wall, floor, or a body surface of a subject P under control of the processing circuitry 34. To give an example, the projector can also perform projection onto an arbitrary plane, object, space, or the like by projection mapping. The processing circuitry 34 may display a composite image described below on the projector, for example.

The memory 33 stores therein various kinds of data under control of the processing circuitry 34. Data storage in the memory 33 will be described below. The memory 33 can also store therein computer programs for the circuitry included in the medical information processing apparatus 30 to implement its functions. The memory 33 is implemented by a semiconductor memory element such as a random access memory (RAM) or a flash memory, a hard disk, or an optical disc, for example. Alternatively, the memory 33 may be implemented by a group of servers (cloud) connected to the medical information processing apparatus 30 via a network.

The processing circuitry 34 executes an acquisition function 34a, an analysis function 34b, and an output function 34c to control the operation of the entire medical information processing apparatus 30. The acquisition function 34a is an example of an acquisition unit. The analysis function 34b is an example of an analysis unit. The output function 34c is an example of an output unit.

The processing circuitry 34 reads a computer program corresponding to the acquisition function 34a from the memory 33 and executes it to acquire a total amount of the contrast agent available for the subject during the procedure, for example. The processing circuitry 34 reads a computer program corresponding to the analysis function 34b from the memory 33 and executes it to acquire an analysis result for each contrast imaging condition about the subject. The processing circuitry 34 reads a computer program corresponding to the output function 34c from the memory 33 and executes it to output information on use of the contrast agent based on the total amount of the contrast agent and the analysis result. The details of the processing by the acquisition function 34a, the analysis function 34b, and the output function 34c will be described below.

In the medical information processing apparatus 30 illustrated in FIG. 1, each processing function is stored in the memory 33 in the form of a computer program that can be executed by a computer. The processing circuitry 34 is a processor reading the computer program from the memory 33 and executing it to implement the function corresponding to each computer program. In other words, the processing circuitry 34 having read the computer program has the function corresponding to the read computer program.

Although the above in FIG. 1 describes a case in which the single processing circuitry 34 implements the acquisition function 34a, the analysis function 34b, and the output function 34c, a plurality of independent processors may be combined with each other to form the processing circuitry 34, and each of the processors may execute the computer program to implement the function. Each processing function of the processing circuitry 34 may be implemented by being distributed or integrated into a single circuit or a plurality of processing circuitries as appropriate.

The processing circuitry 34 may also use a processor of an external apparatus connected via the network NW to perform the functions. The processing circuitry 34 reads the computer program corresponding to each function from the memory 33, executes it, and uses a group of servers (cloud) connected to the medical information processing apparatus 30 via the network NW as a computing resource to implement each function illustrated in FIG. 1, for example.

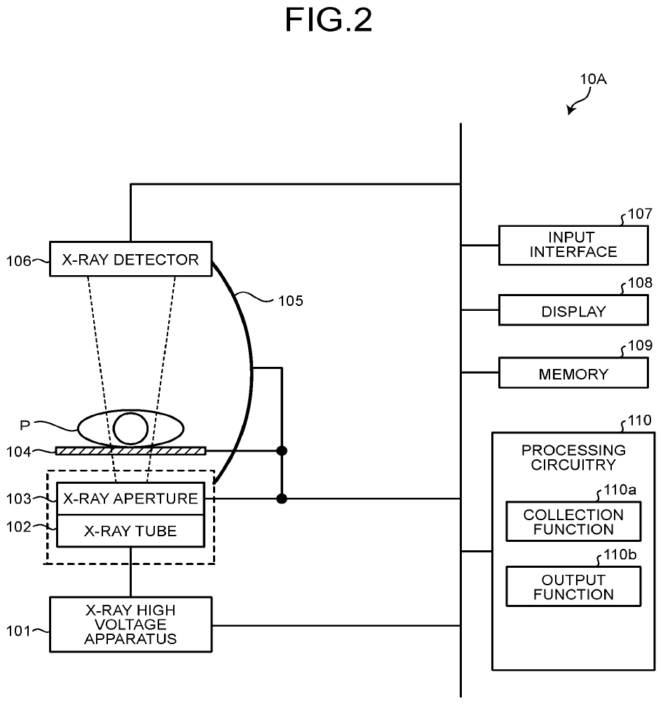

The following describes an example of the configuration of the medical image diagnostic apparatus 10 with reference to FIG. 2. FIG. 2 illustrates an X-ray diagnostic apparatus 10A as an example of the medical image diagnostic apparatus 10. FIG. 2 is a block diagram of an example of the configuration of the X-ray diagnostic apparatus 10A according to the first embodiment.

As illustrated in FIG. 2, the X-ray diagnostic apparatus 10A includes an X-ray high voltage apparatus 101, an X-ray tube 102, an X-ray aperture 103, a couchtop 104, a C arm 105, an X-ray detector 106, an input interface 107, a display 108, a memory 109, and processing circuitry 110.

The X-ray high voltage apparatus 101 supplies high voltage to the X-ray tube 102 under control of the processing circuitry 110. The X-ray high voltage apparatus 101 has an electric circuit such as a transformer and a rectifier and has a high voltage generation apparatus generating high voltage to be applied to the X-ray tube 102 and an X-ray control apparatus controlling output voltage corresponding to X-rays to be applied by the X-ray tube 102, for example. The high voltage generation apparatus may be of the transformer system or of the inverter system.

The X-ray tube 102 is a vacuum tube having a cathode (filament) generating thermoelectrons and an anode (target) generating X-rays upon collision with the thermoelectrons. The X-ray tube 102 applies the thermoelectrons from the cathode toward the anode using the high voltage supplied from the X-ray high voltage apparatus 101 to generate X-rays.

The X-ray aperture 103 has a collimator narrowing the application range of the X-rays generated by the X-ray tube 102 and a filter adjusting the X-rays emitted from the X-ray tube 102.

The collimator of the X-ray aperture 103 has, for example, four slidable aperture blades. The collimator slides the aperture blades to narrow the X-rays generated by the X-ray tube 102 to apply them to the subject P. The aperture blades are plate-shaped members formed of lead or the like and are installed near an X-ray application port of the X-ray tube 102 in order to adjust the application range of the X-rays.

The filter of the X-ray aperture 103, for the purpose of reducing an exposure dose to the subject P and improving the image quality of X-ray images, changes the quality of the X-rays passing therethrough by its material and thickness to reduce a soft ray component, which is easily absorbed by the subject P, and to reduce a high-energy component, which brings about a reduction in the contrast of X-ray images. The filter changes the dose and the application range of the X-rays by its material, thickness, position, or the like and attenuates the X-rays so that the X-rays applied from the X-ray tube 102 to the subject P have preset distribution.

The X-ray aperture 103 has a drive mechanism such as a motor and an actuator and controls the application of the X-rays by operating the drive mechanism under control of the processing circuitry 110 described below, for example. The X-ray aperture 103 adjusts the opening degree of the aperture blades of the collimator by adding drive voltage to the drive mechanism in accordance with a control signal received from the processing circuitry 110 to control the application range of the X-rays to be applied to the subject P, for example. The X-ray aperture 103 adjusts the position of the filter by adding drive voltage to the drive mechanism in accordance with a control signal received from the processing circuitry 110 to control the distribution of the dose of the X-rays to be applied to the subject P, for example.

The couchtop 104 is a bed on which the subject P is placed and is placed on a couch not illustrated. The subject P is not included in the X-ray diagnostic apparatus 10A. The couch has a drive mechanism such as a motor and an actuator and controls the translation and tilt of the couchtop 104 by operating the drive mechanism under control of the processing circuitry 110 described below, for example. The couch adds drive voltage to the drive mechanism in accordance with a control signal received from the processing circuitry 110 to translate or tilt the couchtop 104, for example.

The C arm 105 holds the x-ray tube 102, the X-ray aperture 103, and the X-ray detector 106 such that they face each other with the subject P interposed among them. The C arm 105 has a drive mechanism such as a motor and an actuator and rotates or moves by operating the drive mechanism under control of the processing circuitry 110 described below, for example. The C arm 105 rotates and moves the X-ray tube 102, the X-ray aperture 103, and the X-ray detector 106 with respect to the subject P and controls the application position and the application angle of the X-rays by adding drive voltage to the drive mechanism in accordance with a control signal received from the processing circuitry 110, for example. FIG. 2 describes a case in which the X-ray diagnostic apparatus 10A is a single-plane one as an example, but embodiments are not limited to this example, and it may be a bi-plane one.

The X-ray detector 106 is an X-ray flat panel detector (FPD) having detector elements arranged in a matrix, for example. The X-ray detector 106 detects the X-rays emitted from the X-ray tube 102 and having passed through the subject P and outputs a detection signal corresponding to a detected X-ray dose to the processing circuitry 110. The X-ray detector 106 may be an indirect conversion type detector having a grid, a scintillator array, and an optical sensor array or a direct conversion type detector having a semiconductor element converting incident X-rays into an electric signal.

The input interface 107 can be configured in the same manner as the input interface 31 described above. The input interface 107 receives various kinds of input operations from the user, converts the received input operations into electric signals, and outputs them to the processing circuitry 110, for example.

The display 108 can be configured in the same manner as the display 32 described above. The display 108 displays medical images and GUIs under control of the processing circuitry 110, for example. The X-ray diagnostic apparatus 10A may include a projector instead of or in addition to the display 108.

The memory 109 can be configured in the same manner as the memory 33 described above. The memory 109 stores therein various kinds of data under control of the processing circuitry 110 or stores therein computer programs for the circuitry included in the X-ray diagnostic apparatus 10A to implement its functions, for example.

The processing circuitry 110 executes a collection function 110a and an output function 110b to control the operation of the entire X-ray diagnostic apparatus 10A. The collection function 110a is an example of a collection unit collecting contrast images.

The processing circuitry 110 reads a computer program corresponding to the collection function 110a from the memory 109 and executes it to execute photographing of the subject P and to collect X-ray images, for example. The collection function 110a controls the X-ray high voltage apparatus 101 and adjusts the voltage to be supplied to the X-ray tube 102 to control an X-ray dose to be applied to the subject P and on and off, for example. The collection function 110a controls the operation of the X-ray aperture 103, the couchtop 104, the C arm 105, and the like to control the application range of the X-rays, the dose distribution of the X-rays, the application angle of the X-rays, and the like, for example. Specifically, the collection function 110a controls the operation of the X-ray aperture 103 and adjusts the opening degree of the aperture blades of the collimator to control the application range of the X-rays to be applied to the subject P. The collection function 110a controls the operation of the x-ray aperture 103 and adjusts the position of the filter to control the dose distribution of the X-rays. The collection function 110a rotates or moves the C arm 105 to control the application range and the application angle of the X-rays. The collection function 110a translates or tilts the couchtop 104 to control the application range and the application angle of the X-rays. The collection function 110a generates an X-ray image based on a detection signal received from the X-ray detector 106.

The collection function 110a executes photographing for the subject P with the contrast agent injected and can thereby collect contrast images. The collection function 110a controls the operation of an injector, not illustrated, to inject the contrast agent into the blood vessels of the subject P, thereby collecting contrast images, for example. The injection of the contrast agent may be performed manually by the user.

The processing circuitry 110 reads a computer program corresponding to the output function 110b from the memory 109 and executes it to output the X-ray images collected by the collection function 110a, for example. The output function 110b displays the collected X-ray images on the display 108, for example. The output function 110b also transmits the collected X-ray images to the image storage apparatus 20 via the network NW to be stored therein.

In the X-ray diagnostic apparatus 10A illustrated in FIG. 2, each processing function is stored in the memory 109 in the form of a computer program that can be executed by a computer. The processing circuitry 110 is a processor reading the computer program from the memory 109 and executing it to implement the function corresponding to each computer program. In other words, the processing circuitry 110 having read the computer program has the function corresponding to the read computer program.

Although the above in FIG. 2 describes a case in which the single processing circuitry 110 implements the collection function 110a and the output function 110b, a plurality of independent processors may be combined with each other to form the processing circuitry 110, and each of the processors may execute the computer program to implement the function. Each processing function of the processing circuitry 110 may be implemented by being distributed or integrated into a single circuit or a plurality of processing circuitries as appropriate.

The processing circuitry 110 may also use a processor of an external apparatus connected via the network NW to perform the functions. The processing circuitry 110 reads a computer program corresponding to each function from the memory 109, executes it, and uses a group of servers connected to the X-ray diagnostic apparatus 10A via the network NW as a computing resource to implement each function illustrated in FIG. 2, for example.

The above has described a configuration example of the medical information processing system 1. Under such a configuration, the X-ray diagnostic apparatus 10A collects contrast images in which the blood vessels of the subject P are contrasted while the procedure for the subject P is being performed. The medical information processing apparatus 30 outputs information on use of the contrast agent described below to support appropriate use of the contrast agent in the X-ray diagnostic apparatus 10A.

When the subject P is a patient with ischemic heart disease, for example, endovascular treatment such as percutaneous coronary intervention (PCI) is executed. While such a procedure is being performed, the X-ray diagnostic apparatus 10A collects and displays contrast images as appropriate. This enables the user such as a doctor to proceed with the procedure while keeping track of the shape of a blood vessel and the position of a medical device inserted into the blood vessel.

The contrast agent used by the X-ray diagnostic apparatus 10A to collect contrast images is, for example, an iodine contrast agent with iodine as a main component. The iodine contrast agent injected into the blood vessels of the subject P is metabolized as appropriate by the kidney, but it is undesirable to inject an excessive amount of the iodine contrast agent, and an upper limit is generally set on its use amount. In particular, when the renal function of the subject P is not sufficient, the use amount is required to be limited. The upper limit of the use amount of the iodine contrast agent is determined by, for example, measuring a creatinine value before the procedure and during the procedure and taking into account the weight of the subject P.

Although a smaller use amount of the contrast agent is more preferred, simply reducing the amount of the contrast agent may obscure blood vessels in contrast images, which may affect the efficiency of the procedure. How the contrast agent is appropriately used differs for each subject P and for each scene of the procedure, and it is not easy to determine it.

Given these circumstances, the processing circuitry 34 of the medical information processing apparatus 30 outputs information on use of the contrast agent to make use of the contrast agent more appropriate in a procedure executed with reference to contrast images of the subject P collected by the X-ray diagnostic apparatus 10A. The following describes each processing by the processing circuitry 34 performed before outputting the information on use of the contrast agent.

First, the acquisition function 34a of the processing circuitry 34 acquires the total amount of the contrast agent available for the subject P during the procedure. The acquisition function 34a calculates the available total amount of the contrast agent based on the measurement result of the creatinine value, the weight of the subject P, and the like as described above, for example. Alternatively, the acquisition function 34a may acquire the available total amount of the contrast agent by receiving input operations from the user via the input interface 31.

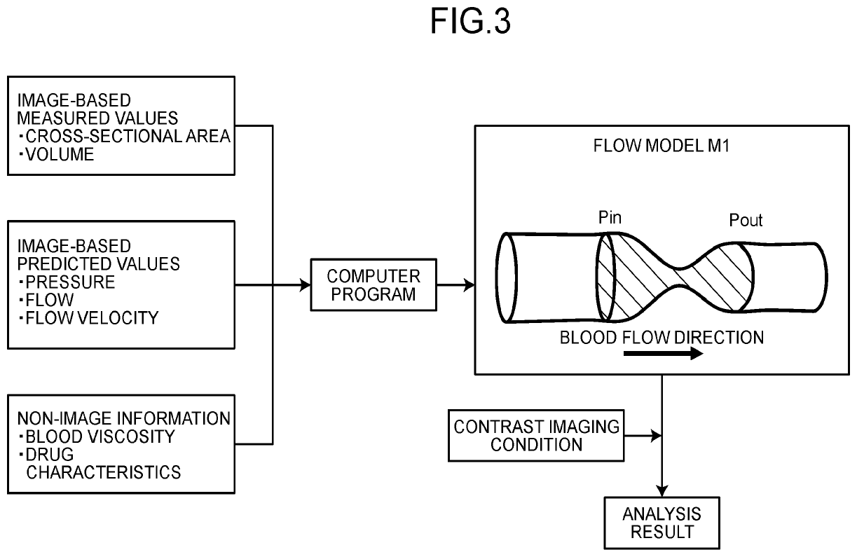

The analysis function 34b generates a flow model M1 illustrated in FIG. 3. The flow model M1 is an estimate of blood flow in the blood vessels of the subject P. That is, the flow model M1 is a model individualized for the subject P.

The flow model M1 is generated by inputting various kinds of parameters such as image-based measured values, image-based predicted values, and non-image information to a certain computer program. The certain computer program is, for example, an application executing fluid simulations based on computational fluid dynamics (CFD).

Examples of the image-based measured values include the cross-sectional area and the volume of the blood vessels. Examples of the image-based predicted values include the pressure, the flow, and the flow velocity of blood flowing through the blood vessels. The image is, for example, a three-dimensional contrast image collected from the subject P. The three-dimensional contrast image may be collected by the X-ray diagnostic apparatus 10A or collected by another kind of modality such as an X-ray CT apparatus or an MRI apparatus. The X-ray diagnostic apparatus 10A can collect a plurality of two-dimensional contrast images from the subject P injected with the contrast agent at different photographing angles while rotating the C arm 105 and reconstruct a three-dimensional contrast image from the two-dimensional contrast images, for example. The three-dimensional contrast image may be acquired directly from the medical image diagnostic apparatus that has collected the three-dimensional contrast image or acquired from the image storage apparatus 20 storing therein the three-dimensional contrast image. The measured values such as a cross-sectional area are examples of luminal structural information of the blood vessels of the subject P.

Examples of non-image information include blood viscosity and drug characteristics. The blood viscosity can be acquired based on an examination result of the blood of the subject P. The analysis function 34b can automatically acquire examination results registered in information management systems such as Hospital Information System (HIS) and Radiology Information System (RIS) and use them to generate the flow model M1, for example.

The drug characteristics are information on drugs to be administered to the subject P. A drug such as a vasodilator or an anticoagulant may be administered to the subject P before the start of the procedure or while the procedure is being performed, for example. When the cross-sectional area described above is a measured value based on a three-dimensional contrast image collected in advance, for example, it is preferable to take into account the effect of the vasodilator in order to estimate an actual cross-sectional area during the procedure. When the blood viscosity described above is based on an examination result of blood collected in advance, for example, it is preferable to take into account the effect of the anticoagulant in order to estimate actual blood viscosity during the procedure. Thus, by including the drug characteristics in input parameters, the flow model M1 can estimate the blood flow in the blood vessels of the subject P during the procedure more accurately.

FIG. 3 is just an example, and various modification are possible for the input information for generating the flow model M1. The analysis function 34b may generate the flow model M1 using shape data indicating the blood vessel shape of the subject P as the luminal structure information instead of or in addition to the image-based measured values, for example. Such shape data can be generated by estimating the position of a blood vessel wall from the three-dimensional contrast image collected from the subject P. Alternatively, the analysis function 34b may use the three-dimensional contrast image itself as the shape data indicating the blood vessel shape of the subject P. In addition, the analysis function 34b may use a hematocrit value or the like as the non-image information.

The flow model M1 in FIG. 3 sets a part with an oblique line pattern of a blood vessel of the subject P (the part from Pin to Pout) as a region of interest (field of view: FOV) and models blood flow in the region of interest. According to the flow model M1, the volume, the flow, the pressure, and the like of the region of interest can be estimated, for example.

Next, the analysis function 34b inputs a contrast imaging condition to the flow model M1 to acquire an analysis result for each input contrast imaging condition. The analysis function 34b acquires, as the analysis result, an estimation result of a contrast image to be collected from the subject P injected with the contrast agent under any contrast imaging condition, for example. The analysis function 34b estimates a contrast image to be collected from the subject P injected with the contrast agent under a contrast imaging condition scheduled for the procedure for the subject P before the start of the procedure for the subject P, for example.

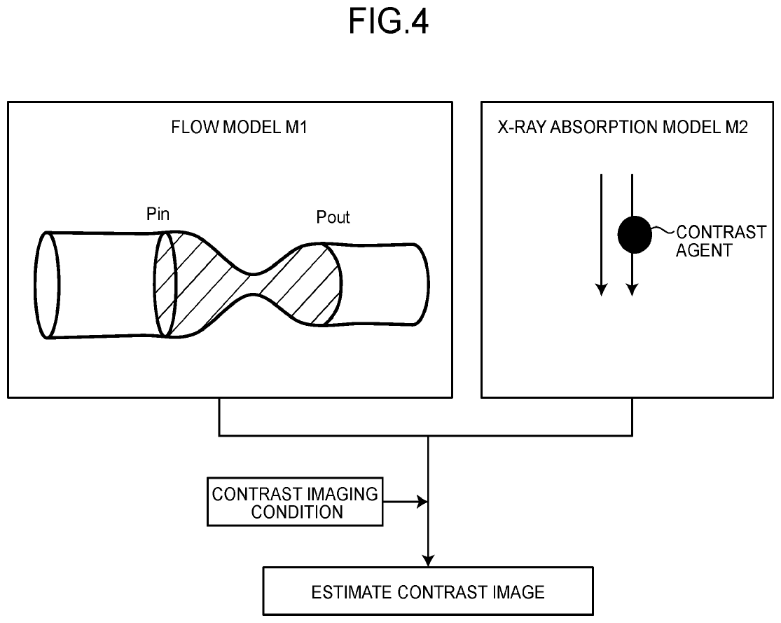

As illustrated in FIG. 4, the analysis function 34b uses the flow model M1 and an X-ray absorption model M2 to acquire the estimation result of the contrast image, for example. The X-ray absorption model M2 is a known model estimating the intensity of X-rays having passed using X-ray energy (a tube voltage value), a ray attenuation coefficient corresponding to the component of the contrast agent, the concentration of the contrast agent, and the like as parameters, for example.

The analysis function 34b inputs various contrast imaging conditions into the flow model M1, such as an injection position at which the contrast agent is injected into the subject P, the concentration of a contrast agent stock solution, a dilution rate, and a pressure, a flow velocity, and a time when the contrast agent is injected, for example. This enables estimation of the distribution of the contrast agent in the region of interest at each point in time after injection of the contrast agent.

The injection position is a position at which the contrast agent is injected into the subject P. In general, the contrast agent is delivered into a blood vessel from the tip of a catheter inserted into the blood vessel. In this case, the injection position is at the tip position of the catheter. The concentration of the contrast agent stock solution is the mass of the contrast agent contained in the contrast agent stock solution per unit volume. The contrast agent is injected into the blood vessel after diluting the contrast agent stock solution at a set dilution rate.

The pressure and flow velocity when the contrast agent is injected are conditions related to the injection rate of the contrast agent. A higher pressure gives a larger flow velocity of the contrast agent when it is delivered from the tip of the catheter into the blood vessel. When the diameter of the catheter is constant, a higher pressure gives a larger amount (flow) of the contrast agent delivered from the tip of the catheter into the blood vessel in a unit time. A longer time when the contrast agent is injected gives a larger amount of the contrast agent delivered from the tip of the catheter into the blood vessel.

Further, the analysis function 34b applies an X-ray application condition and an estimated result of the distribution of the contrast agent in the region of interest to the X-ray absorption model M2 to acquire the estimation result of the contrast image. Specifically, the analysis function 34b can estimate the dose and energy of the X-rays applied from the X-ray tube 102 based on the X-ray application condition. The analysis function 34b can estimate an X-ray attenuation amount occurring when the X-rays having the estimated dose and energy have passed through the subject P containing the contrast agent with the estimated distribution in accordance with the X-ray absorption model M2. This enables the analysis function 34b to estimate the intensity of the X-rays when detected by the X-Ray detector 106 and also the pixel value of each pixel in the contrast image to be collected.

The output function 34c may output the estimation result of the contrast image by the analysis function 34b and provide it to the user such as a doctor. The output function 34c displays the estimated contrast image on the display 32, for example. The analysis function 34b can also estimate the distribution of the contrast agent for each point in time after injection of the contrast agent and estimate the contrast image at each point in time. In this case, the output function 34c can output the estimation result of the contrast image as a moving image showing how the injected contrast agent flows through the blood vessel.

The output function 34c may output a recommended contrast imaging condition under which a contrast image in which a region of attention in the subject P is contrasted can be collected, based on the total amount of the contrast agent available for the subject during the procedure and the estimation result of the contrast image. The region of attention is a region to which the user particularly pays attention during the procedure, such as a stenosis part to be subjected to PCI treatment or a plaque to be treated in chronic total occlusion (CTO), for example. In the PCI treatment, using a balloon, a stent, or the like inserted into the blood vessel, an operation to dilate the stenosis part is performed. In a procedure for CTO, an operation to penetrate the plaque occluding the coronary artery by a device such as a guidewire is performed. The region of attention may be set manually by the user such as a doctor or set automatically by the output function 34c based on the case of the subject P, an examination protocol, or the like. The recommended contrast imaging condition is an example of the information on use of the contrast agent.

The output function 34c evaluates the contrast of the region of attention in the estimated contrast image to set the recommended contrast imaging condition, for example. If the contrast of the region of attention is too low, for example, it is estimated that the blood vessel in the region of attention will not be sufficiently rendered also in the contrast image to be actually collected during the procedure. Thus, the output function 34c sets the recommended contrast imaging condition with the pressure, the flow velocity, the time, or the like when the contrast agent is injected increased, for example, and provides it to the user. If the contrast of the region of attention is too high, the contrast agent may be used excessively and the luminance of the image may be too high, causing artifacts. Thus, the output function 34c sets the recommended contrast imaging condition with the pressure, the flow velocity, the time, or the like when the contrast agent is injected reduced, for example, and provides it to the user.

The appropriate contrast of the region of attention may be preset in accordance with sites, cases, or the like or set based on an operation by the user. For the contrast image collected from the subject P, the user may execute image processing to adjust its contrast so that the region of attention is easily observed, for example. When there is such a contrast image subjected to image processing, the output function 34c can set the recommended contrast imaging condition with the contrast of the contrast image as an appropriate value. The output function 34c may set the recommended contrast imaging condition while updating the appropriate contrast as appropriate in accordance with the region of attention or a photographing angle.

A plaque often develops near the stenosis part of the blood vessel. By performing injection of the contrast agent at a high pressure near the plaque, the plaque may collapse and flow out of the blood vessel, narrowing other blood vessels. Thus, the output function 34c may set the recommended contrast imaging condition so as not to collapse the plaque. The output function 34c may evaluate the hardness of the plaque, and if the plaque is soft and likely to collapse, it may set the recommended contrast imaging condition such that the plaque is less likely to collapse by setting the pressure to inject the contrast agent to be lower, setting the injection position to a position away from the plaque, or the like, for example.

Some contrast imaging conditions, such as the amount of the contrast agent per injection, may be set in advance based on empirical rules or the like for each site or facility. If an injector is used, injection of the contrast agent can also be achieved with high precision in accordance with the set contrast imaging condition. However, it is necessary to consider individual differences for each subject P in order to set the optimum contrast imaging condition. Concerning this matter, the output function 34c can set a more appropriate recommended contrast imaging condition based on the flow model M1 individualized for the subject P.

To give another example, when the contrast agent is injected targeted for a coronary artery, there are known cases in which part of the contrast agent flows toward the aorta, depending on the injection position and the timing with respect to the cardiac phase. Concerning this matter, the analysis function 34b can estimate the contrast image to be collected for each contrast imaging condition such as the injection position or the timing using the flow model M1 individualized for the subject P. The output function 34c can then identify the injection position and the timing minimizing the amount of the contrast agent flowing toward the aorta based on the estimated contrast image and provide them to the user as the recommended contrast imaging condition. Alternatively, the output function 34c can identify the injection position and the timing allowing a moderate amount of the contrast agent to flow toward the aorta based on the estimated contrast image and provide them to the user as the recommended contrast imaging condition.

The output function 34c displays the recommended contrast imaging condition on the display 32 together with the estimation result of the contrast image to be collected from the subject P injected with the contrast agent under the recommended contrast imaging condition, for example. The displayed recommended contrast imaging condition is referred to by the user such as a doctor, and if determined to be appropriate by the user, the recommended contrast imaging condition is set as the contrast imaging condition when the contrast agent is injected into the subject P during the procedure.

The user who refers to the recommended contrast imaging condition displayed on the display 32 executes injection of the contrast agent in accordance with the recommended contrast imaging condition, for example. Alternatively, the user who refers to the recommended contrast imaging condition displayed on the display 32 inputs the recommended contrast imaging condition via the input interface 31 as a contrast imaging setting by the injector included in the medical image diagnostic apparatus 10, and the injector executes injection of the contrast agent in accordance with the input contrast imaging condition.

Alternatively, the output function 34c may transmit the recommended contrast imaging condition to the injector. In this process, the injector retains the transmitted recommended contrast imaging condition as a candidate for the contrast imaging condition. The user selects whether to use the recommended contrast imaging condition via the input interface 31. The user refers to the recommended contrast imaging condition displayed on the display 32 and then selects whether to use the recommended contrast imaging condition, for example. If an input operation to use the recommended contrast imaging condition is performed, the injector sets the recommended contrast imaging condition as an actual contrast imaging setting and also executes injection of the contrast agent in accordance with the set contrast imaging condition.

When the contrast imaging condition is changed, it may be desirable to also change the X-ray application condition in accordance with the changed contrast imaging condition. If the contrast imaging condition is changed such that the contrast agent in the blood vessel becomes thinner, for example, the X-ray energy is preferably reduced in order to improve contrast in the contrast image. Thus, the output function 34c may output the recommended X-ray application condition together with the recommended contrast imaging condition.

The output function 34c outputs at least one of the number of times, a time during which, and an amount with which the contrast agent can be injected into the subject P at the start of the procedure or in the middle of the procedure. The number of times, the time during which, and the amount with which the contrast agent can be injected into the subject P are each an example of the information on use of the contrast agent. In the following, the number of times, the time during which, and the amount with which the contrast agent can be injected into the subject P will also be referred to collectively as availability information of the contrast agent.

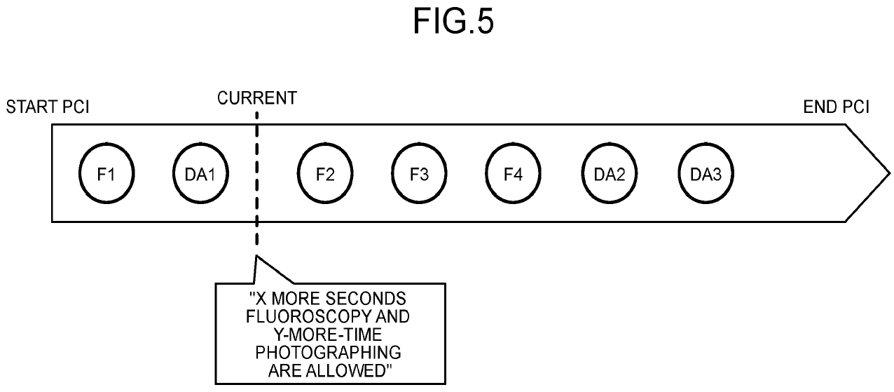

The following describes the availability information of the contrast agent with reference to FIG. 5. FIG. 5 illustrates a use example of the contrast agent in a duration from the start to the end of PCI treatment along a time axis.

In FIG. 5, the PCI treatment is started, and fluoroscopy F1 is first performed. Fluoroscopy F1 is one of the photographing modes in the X-ray diagnostic apparatus 10A, in which a plurality of contrast images (fluoroscopic images) are collected over time and also displayed in real time. In FIG. 5, after Fluoroscopyy F1, Photographing DA1 is performed. Photographing DA1 is one of the photographing modes in the X-ray diagnostic apparatus 10A, in which contrast images with higher image quality than that of the fluoroscopic images are photographed. In Photographing DA1, a larger amount of the contrast agent is injected or a higher dose of X-rays are applied than in Fluoroscopy F1 to collect contrast images with higher image quality, for example. Fluoroscopy F1 is performed continuously for some time, whereas Photographing DA1 is usually performed only once or a few times.

The contrast imaging condition may be set separately for fluoroscopy and photographing. The set contrast imaging condition may be the recommended contrast imaging condition described above, a preset contrast imaging condition, or a contrast imaging condition manually set by the user.

FIG. 5 illustrates a “current” point in time and Fluoroscopy F2, Fluoroscopy F3, Fluoroscopy F4, Photographing DA2, and Photographing DA3 to be performed after the “current”. Fluoroscopy F2, Fluoroscopy F3, Fluoroscopy F4, Photographing DA2, and Photographing DA3 can be estimated from, for example, records of fluoroscopy and photographing in the PCI treatment performed in the past for other subjects having a case similar to that of the subject P.

At the “current” point in time in FIG. 5, a cumulative amount of the contrast agent used in Fluoroscopy F1 and Photographing DA1 is evident. The total amount of the contrast agent available for the subject P during the procedure has been acquired by the acquisition function 34a. The output function 34c then subtracts the cumulative amount of the used contrast agent from the available total amount of the contrast agent and can thereby calculate the amount of the contrast agent that can be injected into the subject P after the “current” point in time. The output function 34c can display the calculated amount of the contrast agent on the display 32 as the information on use of the contrast agent.

The output function 34c can convert the amount of the contrast agent into a time or the number of times. The output function 34c acquires the amount of the contrast agent to be delivered into the blood vessel in a unit time based on the contrast imaging condition during fluoroscopy, divides the amount of the contrast agent that can be injected into the subject P after the “current” point in time by the acquired amount, and can thereby calculate the length of a time during which fluoroscopy can be executed for the subject P after the “current” point in time, for example.

The output function 34c acquires the use amount of the contrast agent per photographing based on the contrast imaging condition during photographing, divides the amount of the contrast agent that can be injected into the subject P after the “current” point in time by the acquired amount, and can thereby calculate the number of times photographing can be executed for the subject P after the “current” point in time, for example.

As illustrated in FIG. 5, the output function 34c can display “X more seconds fluoroscopy and Y-more-time photographing are allowed” at the “current” point in time, for example. This display is an example of the information on use of the contrast agent. “X seconds” and “Y-time” are calculated such that the amount of the contrast agent to be used when “X seconds” fluoroscopy and “Y-time” photographing are performed does not exceed the amount of the contrast agent that can be injected into the subject P after the “current” point in time.

The output function 34c can also output the availability information together with various information. The output function 34c may display the availability information on the display 32, together with the injection position, the cumulative amount of the contrast agent used for the subject P during the procedure, the total amount of the contrast agent available for the subject P during the procedure, and the like, for example.

As described above, the output function 34c can output the availability information as the information on use of the contrast agent. The availability information can be updated as appropriate over time. When Fluoroscopy F2 is further performed after the state illustrated in FIG. 5, for example, the output function 34c can add the amount of the contrast agent used in Fluoroscopy F2 to the cumulative amount, calculate again the availability information of the contrast agent in Fluoroscopy F3 and later, and output it.

The user who refers to the availability information of the contrast agent can consider whether the contrast agent is sufficient until the end of the procedure and fine-tune the contrast imaging condition, the number of seconds of fluoroscopy, the number of times of photographing, and the like. If it is determined that the contrast agent is sufficient until the end of the procedure, for example, the user can proceed with the procedure while better ensuring the placement of a medical device and understanding of a blood vessel shape based on the contrast image by, for example, increasing the number of seconds of fluoroscopy. On the other hand, if it is determined that there is a possibility that the contrast agent is insufficient to end the procedure, the user can proceed with the procedure while keeping in mind, for example, that the number of seconds of fluoroscopy is kept to a minimum length.

The output function 34c may make a use plan of the contrast agent based on the availability information of the contrast agent and provide it to the user. It is possible to estimate the number of seconds of fluoroscopy and the number of times of photographing to be executed during the upcoming procedure, such as Fluoroscopy F2, Fluoroscopy F3, Fluoroscopy F4, Photographing DA2, and Photographing DA3 illustrated in FIG. 5, for example. Thus, the output function 34c can determine whether the contrast agent is sufficient until the end of the procedure based on the availability information of the contrast agent and make the use plan of the contrast agent. The use plan of the contrast agent is an example of the information on use of the contrast agent.

If it is determined that the contrast agent is sufficient until the end of the procedure, for example, the output function 34c can notify the user of being safe to use the contrast agent in a larger amount in part of fluoroscopy and photographing, for example, immediately after the start of the procedure. If it is determined that there is a possibility that the contrast agent becomes insufficient by the end of the procedure, the output function 34c can notify the user of recommendation to save the contrast agent as much as possible. Such notification is an example of the use plan of the contrast agent.

As described above, the medical information processing apparatus 30 according to the first embodiment can make use of the contrast agent during the procedure more appropriate.

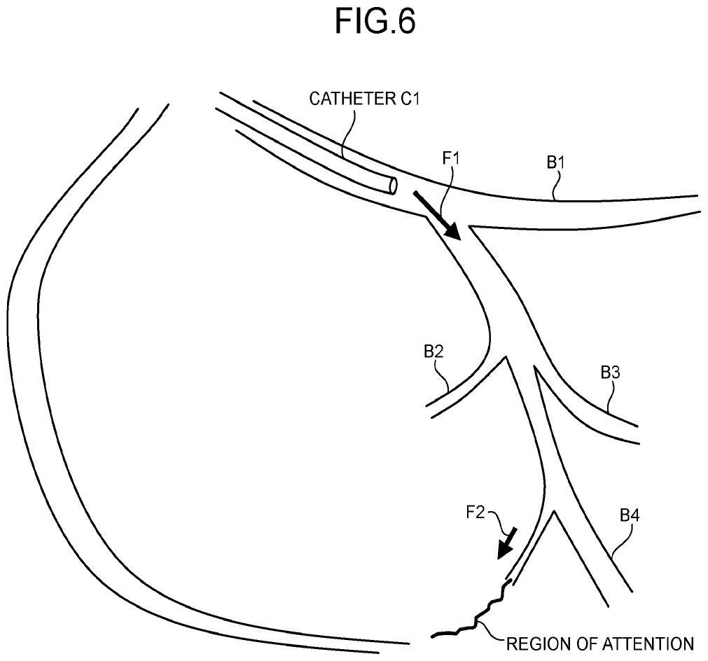

In a procedure for CTO, for example, performed is an operation to penetrate a plaque occluding the coronary artery by a device such as a guidewire while injecting the contrast agent from upstream of the coronary artery to collect a contrast image of the coronary artery. As an example, FIG. 6 illustrates a coronary artery in which the plaque occluding the coronary artery is positioned in the region of attention and also having a branch B1, a branch B2, a branch B3, and a branch B4 between the injection position and the region of attention. A flow F1 near the injection position is divided by these branches, and the flow to the region of attention reduces to a flow F2. It is not easy for the user to intuitively determine a contrast imaging condition to be set under which an appropriate amount of the contrast agent flows into the region of attention.

Concerning this matter, the analysis function 34b can estimate the contrast image to be collected from the subject P when the contrast agent is injected under a certain contrast imaging condition. Specifically, as illustrated in FIG. 3, for example, the contrast image to be collected from the subject P can be estimated based on the information on the individual subject P such as the image-based measured values, the image-based predicted values, and the non-image information. The user who receives the output of such an estimation result of the contrast image can examine whether the contrast imaging condition is appropriate while checking whether the region of attention is sufficiently contrasted even when the blood vessel shape is complex as illustrated in FIG. 6, for example.

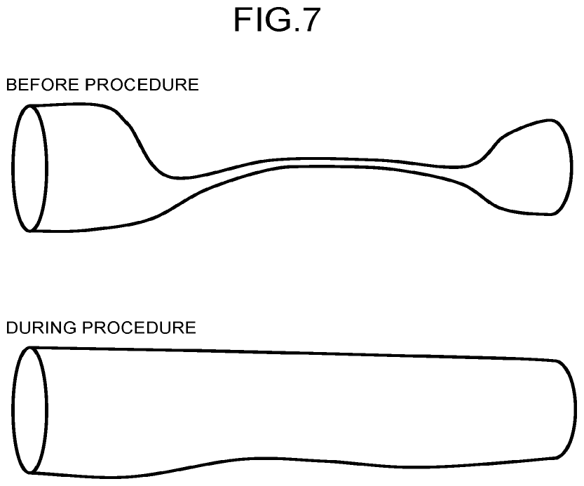

The blood vessel shape may become deformed in the middle of the procedure. As illustrated in FIG. 7, for example, as the effect of the procedure, the stenosis part of the blood vessel may be dilated, and blood flow in the blood vessel may also change. Such deformation of the blood vessel shape can be detected, for example, based on contrast images collected in the fluoroscopy mode or the photographing mode during the procedure. When there is deformation of the blood vessel shape, the analysis function 34b may reflect the deformation in the flow model M1 illustrated in FIG. 3.

The analysis function 34b inputs the cross-sectional area and the volume of the blood vessel after deformation to the certain computer program as the image-based measured values to update the flow model M1, for example. Further, the analysis function 34b estimates again the contrast image to be collected from the subject P based on the updated flow model M1. By thus performing estimation of the contrast image with the deformation of the blood vessel shape during the procedure also taken into account, it is possible to show whether the region of attention is sufficiently contrasted more accurately.

The output function 34c can output the recommended contrast imaging condition under which the contrast image in which the region of attention is contrasted can be collected based on the estimation result of the contrast image. As described above, the estimation of the contrast image is executed based on the information on the individual subject P, not by a uniform simulation such as a training simulator. The estimation of the contrast image can be individualized for each scene of the procedure by, for example, considering the deformation of the blood vessel shape. Thus, the output function 34c can also provide the recommended contrast imaging condition as an appropriate recommended contrast imaging condition that is individualized for each subject P and for each scene.

The output function 34c can output the availability information of the contrast agent and the use plan of the contrast agent based on the availability information at the start of the procedure or during the procedure. This enables the user to use the contrast agent neither without overuse or underuse from the start to the end of the procedure. By receiving provision of the availability information and the use plan, the user can avoid cases in which the contrast agent becomes insufficient by the end of the procedure or use of the contrast agent is avoided more than necessary to reduce the efficiency of the procedure, for example.

The availability information and the use plan of the contrast agent can also be generated based on the recommended contrast imaging condition. In this case, the availability information and the use plan can also be made appropriate, which are individualized for each subject P and for each scene.

A second embodiment describes an example in which when a value of at least one of the number of times, a time during which, and an amount with which the contrast agent is injected into the subject P can be acquired, information on change to the contrast imaging condition that can achieve the value is output to make use of the contrast agent during the procedure more appropriate. In the following, parts overlapping with the description of the first embodiment will be denoted by the same symbols, and descriptions thereof will be omitted.

The case when the value of at least one of the number of times, the time during which, and the amount with which the contrast agent is injected into the subject P can be acquired is a case in which these values are set or a case in which these values can be estimated. When the number of seconds to perform fluoroscopy and the number of times to perform photographing are set in advance, for example, the number of times and the time during which the contrast agent is injected into the subject P are also set values. As in Fluoroscopy F2, Fluoroscopy F3, Fluoroscopy F4, Photographing DA2, and Photographing DA3 in FIG. 5, for example, the number of seconds of fluoroscopy and the number of times of photographing executed during the procedure may be estimated. In the following, the number of times, the time during which, and the amount with which the contrast agent is injected into the subject P will also be referred to collectively as a scheduled use value of the contrast agent.

According to the scheduled use value of the contrast agent and the contrast imaging condition such as pressure or flow velocity when injecting the contrast agent, it is possible to determine whether a scheduled use amount of the contrast agent exceeds the available total amount of the contrast agent. If it is determined that the scheduled use amount of the contrast agent exceeds the available total amount of the contrast agent, the output function 34c outputs information on change to the contrast imaging condition that can achieve the scheduled use value of the contrast agent. The information on change is an example of the information on use of the contrast agent. The details of the information on change will be described below.

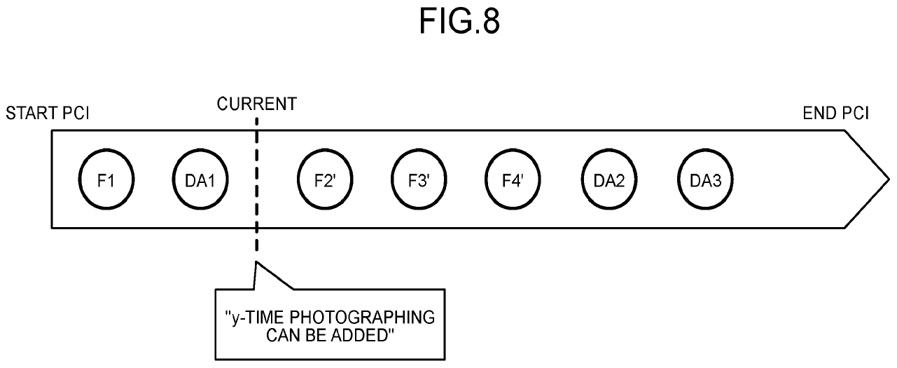

If the user who receives provision of the information on change determines that the change in the condition contained in the information is allowable, he/she changes the contrast imaging condition in accordance with the information. This achieves the scheduled use value of the contrast agent. In FIG. 8, for example, the contrast imaging condition for fluoroscopy has been changed from the state in FIG. 5. Specifically, Fluoroscopy F2, Fluoroscopy F3, and Fluoroscopy F4 are changed to Fluoroscopy F2′, Fluoroscopy F3′, and Fluoroscopy F4′, respectively, with different contrast imaging conditions. This increases the number of times, the time during which, and the amount with which the contrast agent is injected into the subject P and achieves the scheduled use value of the contrast agent. The output function 34c can provide notification of “y-time photographing can be added” together with the contrast imaging condition before and after change, for example.

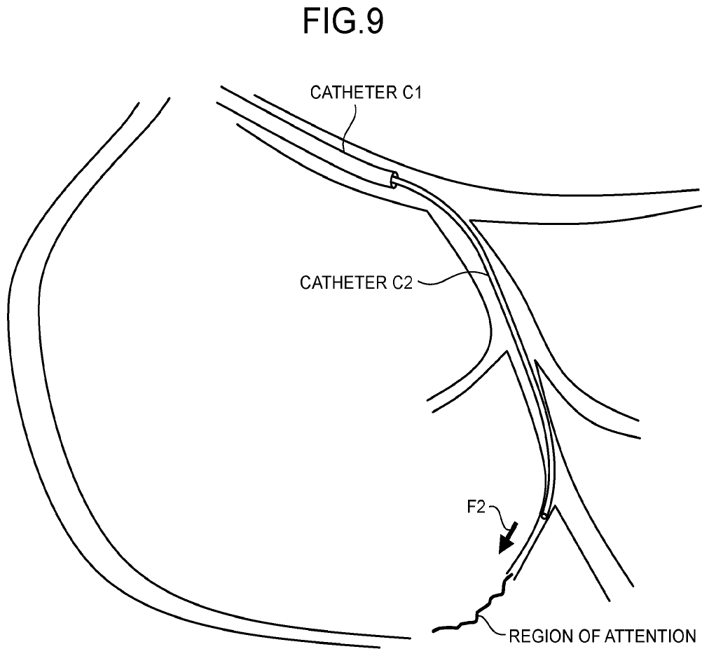

The following describes an example of the information on change with reference to FIG. 9. FIG. 9 describes an example in which the information on change includes a change in the injection position and a change in the medical device for injecting the contrast agent into the subject P. FIG. 9 illustrates a catheter C1 and a catheter C2 as the medical device for injecting the contrast agent into the subject P. FIG. 9 illustrates two positions, or the tip of the catheter C1 and the tip of catheter C2, as the injection position.

By changing the injection position from the position of the tip of catheter C1 to the position of the tip of catheter C2, for example, the distance between the region of attention and the injection position can be reduced. In general, when the region of attention and the injection position are far apart, the contrast agent diffuses in a core wire direction of the blood vessel while flowing through the blood vessel, resulting in a lower concentration of the contrast agent when it reaches the region of attention. In other words, by reducing the distance between the region of attention and the injection position, the concentration of the contrast agent in the region of attention can be increased without changing the use amount of the contrast agent.

Although there are a plurality of branches of the blood vessel between the position of the tip of the catheter C1 and the region of attention, these branches can even be avoided by changing the injection position to the position of the tip of the catheter C2. In general, when there is a branch between the region of attention and the injection position, part of the contrast agent flows into a blood vessel that does not include the region of attention, and thus the concentration of the contrast agent in the region of attention becomes low. In other words, by ensuring that no branch is included between the region of attention and the injection position, the concentration of the contrast agent in the region of attention can be increased without changing the use amount of the contrast agent.

Advancing a catheter to a position close to the region of attention is a workload for the user manipulating the catheter. Using the catheter C2 having a smaller diameter facilitates catheter manipulation within the blood vessel, which can reduce the workload of the user. The catheter having a smaller diameter is also referred to as a microcatheter. That is, in the example in FIG. 9, the information on change includes a change to the microcatheter.

While the microcatheter is easy to manipulate within the blood vessel, its feedable amount of the contrast agent is small because of its smaller diameter. As illustrated in FIG. 9, for example, a small amount of the contrast agent may sufficiently contrast the region of attention when the region of attention and the injection position are close to each other or the like, but there may be a case in which the contrast agent in the feedable amount by the microcatheter cannot sufficiently contrast the region of attention. The output function 34c preferably includes the change to the microcatheter in the information on change after determining whether the contrast agent in the feedable amount by the microcatheter can sufficiently contrast the region of attention. The analysis function 34b estimates the contrast image to be collected from the subject P injected with the contrast agent in the feedable amount by the microcatheter, for example. The output function 34c then determines whether the region of attention is sufficiently contrasted based on the estimated contrast image and, if determining that the region of attention is sufficiently contrasted, includes the change to the microcatheter in the information on change.

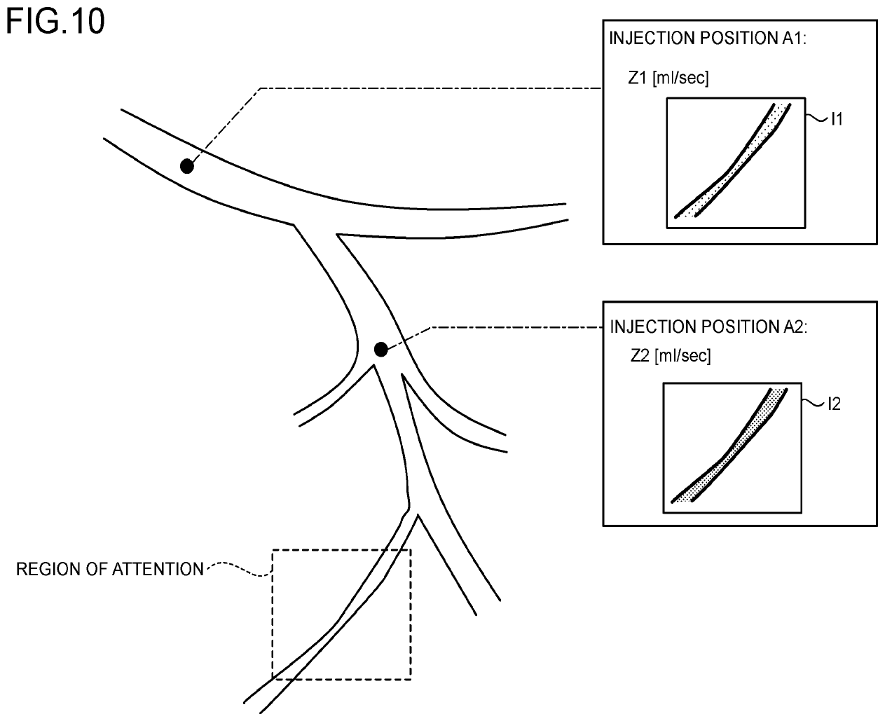

FIG. 10 illustrates a display example of the information on change. In FIG. 10, an injection position A1, which is the injection position before change, and an injection position A2, which is the injection position after change, are displayed on a contrast image in which a blood vessel of the subject P is contrasted. The contrast image displaying the injection position A1 and the injection position A2 may be an image collected by the X-ray diagnostic apparatus 10A or an image collected by another kind of modality such as an X-ray CT apparatus or an MRI apparatus.

In FIG. 10, for each position of the injection position A1 and the injection position A2, an injection rate and an image of a range corresponding to the region of attention in the estimation result of the contrast image to be collected from the subject P injected with the contrast agent at that injection rate are displayed. More specifically, an image I1 in FIG. 10 is an image of the region of attention estimated when the contrast agent is injected from the injection position A1 at an injection rate of “Z1 [m1/sec]”. An image I2 is an image of the region of attention estimated when the contrast agent is injected from the injection position A2 at an injection rate of “Z2 [m1/sec]”.

The injection rate “Z2 [m1/sec]” in FIG. 10 may be the same as the injection rate “Z1 [m1/sec]” or smaller than the injection rate “Z1 [m1/sec]”. Because the injection position A2 is closer to the region of attention than the injection position A1 is, it is possible to contrast the blood vessel in the image I2 to the same degree as or a greater degree than in the image I1 even when the injection rate “Z2 [m1/sec]” is smaller than the injection rate “Z1 [m1/sec]”. The difference between the injection rate “Z1 [m1/sec]” and the injection rate “Z2 [m1/sec]” indicates the amount of the contrast agent reduced by changing the injection position.

The user who refers to the display in FIG. 10 can determine whether to allow the change in the injection position by comprehensively considering the viewability of the image I1 and the image I2 each, the amount of the contrast agent reduced by changing the injection position, the workload of inserting the catheter to the position of the injection position A2, and the like.

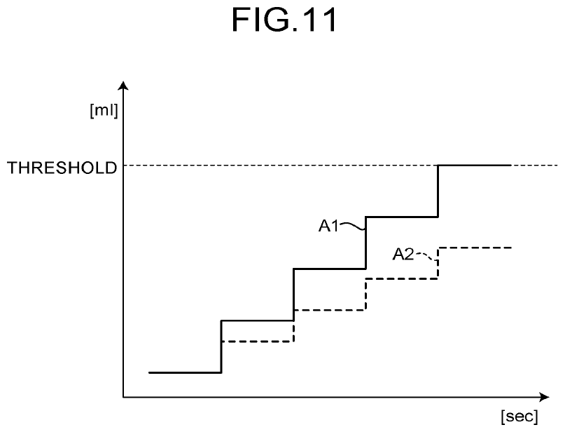

The graph in FIG. 11 is shown as another display example of the amount of the contrast agent reduced by changing the injection position. The horizontal axis in FIG. 11 shows an elapsed time (sec) after the start of the procedure. The vertical axis in FIG. 11 shows a cumulative amount of the contrast agent when the contrast agent is injected from each position of the injection position A1 and the injection position A2 in FIG. 10. In the case of the injection position A2, the injection rate can be set to be lower than in the case of the injection position A1, and thus the pace at which the cumulative amount of the contrast agent increases is slower. The difference in the vertical axis between the graph corresponding to the injection position A1 and the graph corresponding to the injection position A2 indicates the amount of the contrast agent reduced by changing the injection position at each point in time after the start of the procedure.

In FIG. 11, the total amount of the contrast agent available for the subject P during the procedure is shown as a “threshold”. The user who refers to the display in FIG. 10 can consider whether the procedure can be ended before the cumulative amount of the contrast agent reaches the threshold and determine whether to allow the change in the injection position. The display in FIG. 11 may be done independently or done together with the display in FIG. 10.

As examples of the information on change, the change in the injection position and the change in the medical device for injecting the contrast agent have been described. However, embodiments are not limited to these examples. The output function 34c may output a reduction in the concentration of the contrast agent to be injected into the subject P as the information on change to the contrast imaging condition that can achieve the scheduled use value of the contrast agent, for example.

That is, when the contrast agent concentration is reduced, the contrast of the region of attention reduces, but this reduction in contrast may be allowable to the user. The output function 34c, for each contrast agent concentration before and after change, displays a contrast image estimated when the contrast agent is injected at each contrast agent concentration, for example. In this process, the output function 34c may together display the amount of the contrast agent reduced by reducing the contrast agent concentration, as well as the number of seconds of fluoroscopy and the number of times of photographing that become executable in an additional manner by reducing the contrast agent concentration. The user who refers to such a display can determine whether to allow the change by comparing the estimated contrast images and considering whether the contrast reduction is allowable.

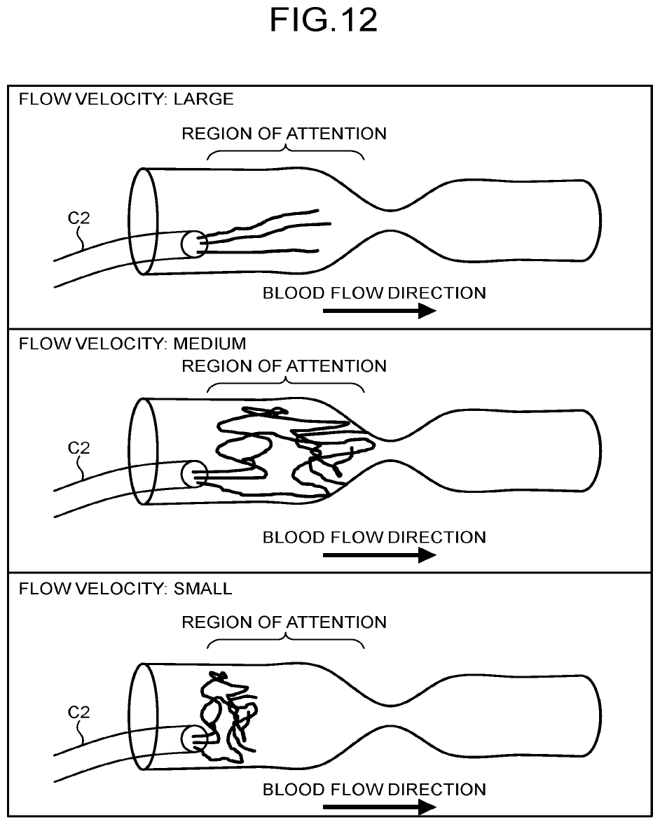

FIG. 12 illustrates another example of the information on change. FIG. 12 illustrates a case in which the distance between the injection position (the tip of the catheter C2) and the region of attention is close. The contrast agent preferably diffuses widely within the region of attention as illustrated in the middle panel of FIG. 12, and for this purpose, the flow velocity of the contrast agent during injection is required to be set appropriately.

Specifically, if the flow velocity of the contrast agent during injection is too large, as illustrated in the upper panel of FIG. 12, the injected contrast agent may pass through the region of attention without diffusing into the blood. This may result in the region near the wall of the blood vessel in the region of attention not being contrasted in the contrast image to be collected.

On the other hand, if the flow velocity of the contrast agent during injection is too small, as illustrated in the lower panel of FIG. 12, the injected contrast agent does not necessarily diffuse sufficiently in a blood flow direction, although it diffuses to the region near the wall of the blood vessel. This may result in part of the region of attention in the blood flow direction not being contrasted in the image to be collected. Alternatively, injection of the contrast agent is continued for a longer time in order to diffuse the contrast agent widely in the blood flow direction, which may increase the use amount of the contrast agent.

The analysis function 34b, for a plurality of contrast imaging conditions with different flow velocities, estimates contrast images to be collected from the subject P injected with the contrast agent under the respective contrast imaging conditions. The output function 34c identifies, from among a plurality of estimated contrast images, a contrast image in which the contrast agent is widely diffused in the region of attention and also identifies a flow velocity corresponding to the identified contrast image. The output function 34c then displays the identified flow velocity as the information on change. In this process, the output function 34c may together display the estimated contrast image and the use amount of the contrast agent reduced by changing the flow velocity.

Apart from the examples described above, the output function 34c can output the information on change together with various information. The output function 34c may display the information on change on the display 32, together with the cumulative amount of the contrast agent used for the subject P during the procedure, the total amount of the contrast agent available for the subject P during the procedure, for example.

As described above, the output function 34c according to the second embodiment outputs, for the value of at least one of the number of times, the time during which, and the amount with which the contrast agent is injected into the subject P, the information on change to the contrast imaging condition that can achieve the value. This enables use of the contrast agent to be continued by appropriately changing the contrast imaging condition even when the cumulative use amount of the contrast agent approaches the available total amount of the contrast agent and there is a possibility that use of the contrast agent cannot be continued until the end of the procedure, for example, in the middle of the procedure. By changing the injection position to a position close to the region of attention, the contrast image in which the region of attention is contrasted can be collected with a smaller amount of the contrast agent, for example. This increases the number of seconds during which fluoroscopy is possible and the number of times photographing is possible and can continue use of the contrast agent until the end of the procedure.

A third embodiment describes an example of performing feedback to the medical image diagnostic apparatus 10 based on the analysis result by the analysis function 34b as an example of outputting the information on use of the contrast agent. In the following, parts overlapping with the description of the first and second embodiments will be denoted by the same symbols, and descriptions thereof will be omitted.

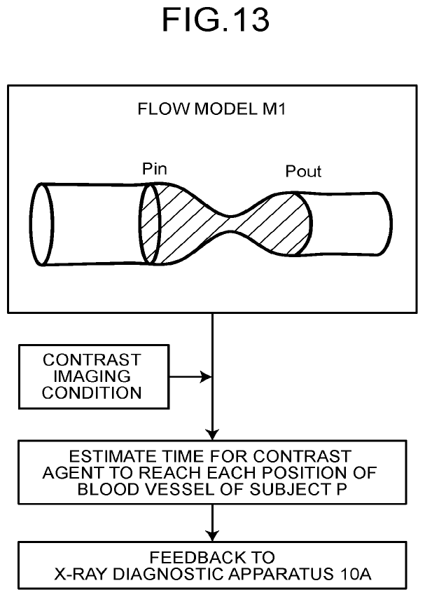

FIG. 13 illustrates an example of processing according to the third embodiment. As in the case described in FIG. 4, the analysis function 34b can estimate the distribution of the contrast agent for each point in time after injection of the contrast agent based on the flow model M1 and estimate the contrast image at each point in time. That is, the analysis function 34b can generate a moving image showing how the injected contrast agent flows through the blood vessel. With such a moving image, the output function 34c can estimate the time for the contrast agent to reach each position of the blood vessel of the subject P after the contrast agent is injected and perform feedback to the X-ray diagnostic apparatus 10A based on the estimated result.

The output function 34c outputs control information for the couchtop 104 included in the X-ray diagnostic apparatus 10A as the information on use of the contrast agent, for example. Specifically, in the case of collecting contrast images of a wide range of blood vessels, such as the inferior vena cava, for example, a technique is known to sequentially change an X-ray application range so as to follow the flow of the contrast agent by moving the couchtop 104. In this process, it is preferable to set the timing to start moving the couchtop 104 and the moving speed of the couchtop 104 in consideration of individual differences.

Concerning this matter, the output function 34c can estimate the time for the contrast agent to reach each position of the blood vessel of the subject P based on the model M1 individualized for the subject P and set the control information such as the timing to start moving the couchtop 104 and the moving speed of the couchtop 104. The output function 34c then feeds back the set control information to the X-ray diagnostic apparatus 10A, thereby enabling the X-ray diagnostic apparatus 10A to execute collection of contrast images performed while moving the couchtop 104 under the appropriate control information individualized for the subject P.

The output function 34c outputs a photographing timing in the X-ray diagnostic apparatus 10A as the information on use of the contrast agent, for example. Specifically, as illustrated in FIG. 6, for example, there may be a distance between the injection position and the region of attention. From the viewpoint of reducing a radiation dose to the subject P, a region to be photographed is preferably limited to a narrow range including the region of attention, and for this purpose, the photographing timing may be appropriately set in accordance with the time required for the contrast agent to reach the region of attention after being injected at the injection position. In this process, the time required for the contrast agent to reach the region of attention is preferably set in consideration of individual differences.

Concerning this matter, the output function 34c can estimate the time for the contrast agent to reach each position of the blood vessel of the subject P based on the model M1 individualized for the subject P and set the time required for the contrast agent to reach the region of attention after being injected at the injection position. The output function 34c then feeds back the set photographing timing to the X-ray diagnostic apparatus 10A, thereby enabling the X-ray diagnostic apparatus 10A to execute collection of the contrast image for the region of attention at the appropriate photographing timing individualized for the subject P.

The control information for the couchtop 104 and the photographing timing of the X-ray image have been described as examples of the feedback to the medical image diagnostic apparatus 10, but embodiments are not limited to these examples. The output function 34c may transmit X-ray conditions such as the tube voltage and tube current of the X-ray tube 102 to the X-ray diagnostic apparatus 10A, for example.

The analysis function 34b estimates the distribution of the contrast agent after injection of the contrast agent based on the flow model M1 and also estimates the contrast image to be obtained for each of the X-ray conditions, for example. The output function 34c then sets the X-ray conditions that can produce a contrast image with appropriate contrast and feeds them back to the X-ray diagnostic apparatus 10A. The appropriate contrast may be preset in accordance with sites, cases, or the like or set based on an operation by the user as described above.

As a method for setting the X-ray conditions to obtain the appropriate contrast, auto brightness control (ABC) is conventionally known. ABC sequentially changes the X-ray conditions based on a collected X-ray image to automatically adjust the contrast of the X-ray image. However, ABC cannot set appropriate X-ray conditions at the start of collection of the X-ray image, or at least in the first frame. Concerning this matter, the output function 34c can set the X-ray conditions to obtain the appropriate contrast from the start of collection of the X-ray image based on the model M1. In turn, the number of frames of the X-ray image to be collected can be reduced, and the use amount of the contrast agent and the exposure dose can be reduced.

The first to third embodiments have been described; embodiments may be executed with various modifications added other than the embodiments described above.

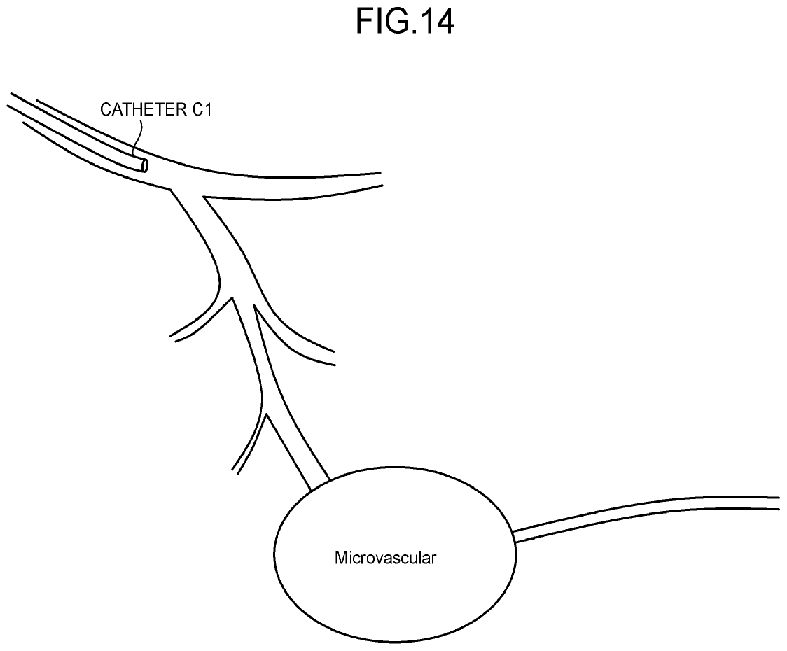

As illustrated in FIG. 14, the analysis function 34b may analyze a discharge period (washout ability) of the contrast agent in microvessels (microvasculature) for each contrast imaging condition, for example. Specifically, when the contrast agent is injected into a site having microvessels such as the myocardium, it is preferable not to inject the contrast agent in a period until the contrast agent is discharged from the microvessels. Although the washout ability of the contrast agent has individual differences by the subject P, the output function 34c can estimate the washout ability based on the model M1 individualized for the subject P. The output function 34c can thus output, as the information on use of the contrast agent, the amount of the contrast agent per injection, an interval for injecting the contrast agent, the maximum amount of the contrast agent that can be injected per unit time, and the like.

The embodiments described above describe a case of collecting the two-dimensional contrast image. However, embodiments are not limited to this example and can be applied to cases in which a three-dimensional contrast image is collected in the same manner. The X-ray diagnostic apparatus 10A can collect a plurality of two-dimensional contrast images with different photographing angles while rotating the C arm 105 and reconstruct a three-dimensional contrast image from the two-dimensional contrast images, for example. During the collection of such a three-dimensional image also, similarly, the medical information processing apparatus 30 can make use of the contrast agent during the procedure more appropriate.

When the X-ray diagnostic apparatus 10A collects the three-dimensional contrast image, it takes some length of a photographing time to complete the collection of the two-dimensional contrast images by rotating the C arm 105. To acquire the three-dimensional contrast image for the region of attention, it is necessary to keep a state in which the region of attention is filled with the contrast agent from the start to the end of the photographing time. This is because if the region of attention is not contrasted in some of the two-dimensional contrast images, artifacts will occur in the three-dimensional contrast image to be reconstructed. Conventionally, during the collection of the three-dimensional contrast image, there has been a tendency to set a longer injection time for the contrast agent in order to avoid the occurrence of artifacts more reliably. Concerning this matter, the medical information processing apparatus 30 of the embodiments can set a more appropriate contrast imaging condition individualized for the subject P. In turn, the occurrence of artifacts can be avoided while shortening the injection time for the contrast agent, and the use amount of the contrast agent during the collection of the three-dimensional contrast image can be reduced.

The embodiments described above describe a case in which the contrast image is collected by the X-ray diagnostic apparatus 10A. However, embodiments are not limited to this example and can also be applied to cases in which the contrast image is collected by other kinds of modalities such as an X-ray CT apparatus and an MRI apparatus in the same manner.

The embodiments described above describe the PCI treatment and the treatment for CTO as examples of the procedure performed while collecting the contrast image. However, embodiments are not limited to these examples.

The embodiments described above can also be applied to treatment for the abdominal aorta, for example. Specifically, when the subject P is a patient having an abdominal aortic aneurysm, performed is a procedure in which a stent graft made to mimic the shape of the abdominal aorta of the subject P is indwelled in the abdominal aorta. After indwelling the stent graft, a blood leak may occur around the stent graft. It is known that controlling such a leak improves prognosis, and thus a follow-up examination after indwelling the stent graft is important.

In the follow-up examination after indwelling the stent graft, the contrast agent is flowed through the abdominal aorta to collect a contrast image. While it is necessary to inject a large amount of the contrast agent in order to contrast the abdominal aorta, there are cases in which the presence or absence of the leak cannot be determined if the injection amount of the contrast agent is insufficient. That is, during the collection of the contrast image of the abdominal aorta, it is not desirable to easily increase an injection amount of the contrast agent, which is originally a large injection amount, but it is even less desirable to decrease the injection amount of the contrast agent to too small and to recollect the contrast image. Concerning this matter, the output function 34c can appropriately set the recommended contrast imaging condition based on the flow model M1 individualized for the subject P and collect the contrast image of the abdominal aorta using a necessary and sufficient amount of the contrast agent. When a medical device such as a stent graft is indwelled in the blood vessel of the subject P, the analysis function 34b can generate the flow model M1 with the medical device as the luminal structure information.

Further, the output function 34c may assume the presence or absence and the amount of a leak to set the recommended contrast imaging condition. Specifically, when there is a leak, the contrast image with contrast with which the leak can be visually recognized is required to be collected. The output function 34c may set the recommended contrast imaging condition such that such contrast is achieved in the contrast image to be collected. Even if there is an endoleak, influence on the prognosis may be negligible depending on its amount. Thus, the output function 34c may set an endoleak amount having influence on the prognosis and set the recommended contrast imaging condition such that contrast with which the set amount of an endoleak can be visually recognized can be obtained. For the endoleak amount having influence on the prognosis, a literature value may be used or a setting by the user such as a doctor may be received.

There may be a time lag between when the contrast agent reaches the abdominal aorta and when the leak is contrasted. Thus, to cover such a time lag, the output function 34c may perform feedback to the medical image diagnostic apparatus 10 so as to increase the time during which the contrast agent is injected or increase the photographing time.

In addition, the above embodiments can be applied to procedures for various cases such as cerebral infarction and cerebral arteriovenous malformation (AVM), for example. In the case of cerebral infarction, for example, the above embodiments can be applied with the position of a cerebral blood vessel occluded by a thrombus or the like as a site of attention. The AVM portion of the cerebral blood vessel is not represented on an image even using the contrast agent, and its exact shape cannot necessarily be grasped. The analysis function 34b may perform modeling to generate the flow model M1 for portions the shape of which cannot be grasped from the image, such as AVM.

The embodiments described above describe a case in which the information on use of the contrast agent is displayed on the display 32 as a specific example when the output function 34c outputs the information on use of the contrast agent. However, embodiments are not limited to these examples. The output function 34c may transmit the information on use of the contrast agent to another apparatus, and the information on use of the contrast agent may be displayed on the other apparatus, for example. The output function 34c transmits the information on use of the contrast agent to the X-ray diagnostic apparatus 10A via the network NW, for example. In this case, the X-ray diagnostic apparatus 10A can display the information on use of the contrast agent on the display 108.

The embodiments described above describe a case in which the various kinds of functions such as the acquisition function 34a, the analysis function 34b, and the output function 34c are implemented in the processing circuitry 34 included in the medical information processing apparatus 30. However, embodiments are not limited to these examples. The same functions as the acquisition function 34a, the analysis function 34b, and the output function 34c may be implemented in the processing circuitry included in the medical image diagnostic apparatus 10, such as the processing circuitry 110 included in the X-ray diagnostic apparatus 10A, for example.

The term “processor” used in the above description means a circuit such as a CPU, a graphics processing unit (GPU), an application specific integrated circuit (ASIC), a programmable logic device (a simple programmable logic device (SPLD), a complex programmable logic device (CPLD), or a field programmable gate array (FPGA), for example), for example. When the processor is a CPU, for example, the processor reads a computer program stored in a storage circuit and executes it to implement a function. On the other hand, when the processor is an ASIC, for example, in place of storing the computer program in the storage circuit, the function is directly embedded in the circuitry of the processor as a logic circuit. Each processor of the embodiments is not limited to being configured as a single circuit for each processor but may also be configured as one processor by combining a plurality of independent circuits to implement its functions. Further, a plurality of components in each drawing may be integrated into one processor to implement their functions.

The components of each apparatus according to the embodiments described above are functionally conceptual ones and do not necessarily need to be physically configured as illustrated in the drawing. That is to say, the specific form of the dispersion and integration of each apparatus is not limited to the one illustrated in the drawing, but the whole or part thereof can be configured in a functionally or physically distributed and integrated manner in any unit in accordance with various kinds of loads, use conditions, and the like. Further, the whole or any part of the processing functions performed by each apparatus can be implemented by a CPU and a computer program that is analyzed and executed by the CPU or be implemented as hardware by wired logic.

The method of medical information processing described in the embodiments described above can be implemented by executing a medical information processing program prepared in advance on a computer such as a personal computer or a workstation. This medical information processing program can be distributed via a network such as the Internet. This medical information processing program can also be executed by being recorded on a computer-readable, non-transitory recording medium such as a hard disk, a flexible disk (FD), a compact disc read only memory (CD-ROM), magneto-optical (MO), or a digital versatile disc (DVD) and being read from the recording medium by a computer.

At least one of the embodiments described above can make use of the contrast agent during the procedure more appropriate.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to
acquire a total amount of a contrast agent available for a subject during a procedure,
acquire an analysis result for each contrast imaging condition about the subject, and
output information on use of the contrast agent based on the total amount of the contrast agent and the analysis result.

2. The medical information processing apparatus according to claim 1, wherein the information on use of the contrast agent includes at least one of a number of times, a time during which, and an amount with which the contrast agent is able to be injected into the subject.

3. The medical information processing apparatus according to claim 2, wherein the information on use of the contrast agent includes a use plan of the contrast agent based on at least one of the number of times, the time during which, and the amount with which the contrast agent is able to be injected into the subject.

4. The medical information processing apparatus according to claim 2, wherein the processing circuitry outputs at least one of the number of times, the time during which, and the amount with which the contrast agent is able to be injected into the subject together with at least one of an injection position at which the contrast agent is injected into the subject, a cumulative amount of the contrast agent used for the subject during the procedure, and the total amount.

5. The medical information processing apparatus according to claim 1, wherein the information on use of the contrast agent includes, when a value of at least one of a number of times, a time during which, and an amount with which the contrast agent is injected into the subject is able to be acquired, information on change to the contrast imaging condition under which the value can be achieved.

6. The medical information processing apparatus according to claim 5, wherein the information on change includes a change in an injection position at which the contrast agent is injected into the subject.

7. The medical information processing apparatus according to claim 5, wherein the processing circuitry displays, as the information on change, an injection position before change and at least one injection position after change on a contrast image in which a blood vessel of the subject is contrasted and further displays an amount of the contrast agent to be reduced by changing the injection position in association with the at least one injection position after change.

8. The medical information processing apparatus according to claim 5, wherein the information on change includes a reduction in a concentration of the contrast agent to be injected into the subject.

9. The medical information processing apparatus according to claim 5, wherein the information on change includes a change in a medical device for injecting the contrast agent into the subject.

10. The medical information processing apparatus according to claim 5, wherein the processing circuitry outputs the information on change together with an amount of the contrast agent to be reduced by changing the contrast imaging condition.

11. The medical information processing apparatus according to claim 5, wherein the processing circuitry outputs the information on change together with information corresponding to each contrast imaging condition before and after change, the information being at least either at least one of a number of times, a time during which, and an amount with which the contrast agent is able to be injected into the subject or an injection position at which the contrast agent is injected into the subject.

12. The medical information processing apparatus according to claim 5, wherein the processing circuitry outputs the information on change together with at least either a cumulative amount of the contrast agent used for the subject during the procedure or the total amount.

13. The medical information processing apparatus according to claim 1, wherein the information on use of the contrast agent includes a recommended contrast imaging condition under which a contrast image in which a region of attention in the subject is contrasted is able to be collected.

14. The medical information processing apparatus according to claim 13, wherein the recommended contrast imaging condition include at least one condition of a pressure, a flow velocity, and a time when the contrast agent is injected into the subject.

15. The medical information processing apparatus according to claim 13, wherein the processing circuitry transmits the recommended contrast imaging condition to an injector executing injection of the contrast agent into a blood vessel of the subject.

16. The medical information processing apparatus according to claim 1, wherein the analysis result includes an estimation result of a contrast image to be collected from the subject injected with the contrast agent under the contrast imaging condition.

17. The medical information processing apparatus according to claim 1, wherein the processing circuitry outputs, as the information on use of the contrast agent, to an X-ray diagnostic apparatus collecting an X-ray image from the subject injected with the contrast agent, information including at least one of control information for a couchtop included in the X-ray diagnostic apparatus and a photographing timing of the X-ray image.

18. The medical information processing apparatus according to claim 1, wherein the processing circuitry acquires the analysis result based on luminal structure information of a blood vessel of the subject, a position of a region of interest, and an injection position at which the contrast agent is injected into the subject.

19. A medical image diagnostic apparatus comprising processing circuitry configured to
collect a contrast image of a subject,
acquire, for a contrast agent used for collection of the contrast image, a total amount of the contrast agent available for the subject during a procedure,
acquire an analysis result for each contrast imaging condition about the subject, and
output information on use of the contrast agent based on the total amount of the contrast agent and the analysis result.

20. A non-transitory computer readable medium comprising instructions that cause a computer to execute:
acquiring a total amount of a contrast agent available for a subject during a procedure,
acquiring an analysis result for each contrast imaging condition about the subject, and
outputting information on use of the contrast agent based on the total amount of the contrast agent and the analysis result.
